(19) United States
(12) Patent Application Publication
(10) Pub. No.: US 2012/0016037 A1
(43) Pub. Date: Jan. 19, 2012

(54) Method for prevention and treatment of Alzheimers

(76) Inventor: Frank Toppo, Pahrump, NV (US)

(21) Appl. No.: 11/491,718

(22) Filed: Jul. 24, 2006

(51) Int. Cl. A61K 31/05; A61P 25/28

(52) U.S. Cl. 514/733

(57) ABSTRACT

Elderly individuals are administered dosages of Trans-resveratrol totaling between about 250 mg and 1 gram per day. Typically about 500 mg of Trans-resveratrol are orally administered to such individuals on a daily basis. As a result of the chemical action of Trans-resveratrol in the bodies of the subjects, Alzheimer's disease is prevented, or if already present, advancement is retarded and the existing mentally debilitating conditions of Alzheimer's disease are treated. A subject receiving the Trans-resveratrol experiences a reduction or illumination of the effects of Alzheimer's disease.

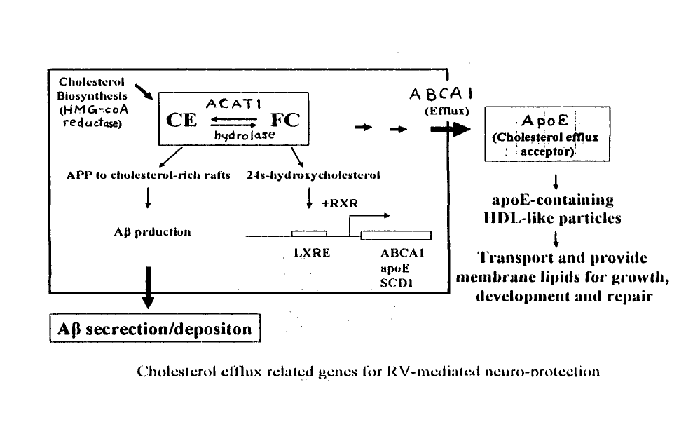

### BACKGROUND OF THE INVENTION

1. Field of the Invention

The present invention relates to a method of prevention, retardation and treatment of Alzheimer's disease by the administration to a human subject of Trans-resveratrol.

2. Description of the Prior Art

It has been established that Trans-resveratrol can be quite beneficial to a human subject in the treatment of a high level all of light density lipoproteins (LDL) in a subject's blood level, as well as a low level of heavy density lipoproteins (HDL) in a subject's blood level. Such a treatment is set forth in prior U.S. Pat. No. 6,048,903 which is hereby incorporated by reference in its entirety.

### SUMMARY OF THE INVENTION

It has now been discovered that the administration of Trans-resveratrol to a human subject is also beneficial in treating, suppressing, retarding and preventing the disease known as Alzheimer's disease. This disease is associated with the aging process and has no known cure. Alzheimer's disease affects the mental faculties of a subject and is typified by the loss of memory, particularly short-term memory and a degradation of the human mental capabilities normally present in all adults. Alzheimer's disease typically commences with a loss of short-term memory and a loss of mental relation by the subject to others and his or her surroundings. As Alzheimer's disease advances the subject loses both short-term and long-term memory and is no longer cogent. The subject is no longer able to intelligently interact with other people or perform simple tasks necessary for personal care. Due to the increased longevity of the average human life span in current times, Alzheimer's disease affects a greater portion of the elderly population each year.

We have discovered that the administration of Trans-resveratrol to human subjects very significantly retards the advancement of Alzheimer's disease and actually partially reverses the debilitating mental deterioration that is characteristic of Alzheimer's disease.

In one broad aspect the present invention may be considered to be a method of treating a human subject for Alzheimer's disease comprising administering to the subject between about 200 mg and about 1 gram of Trans-resveratrol daily. The Trans-resveratrol is preferably administered orally and in a dosage of about 500 mg on a daily basis.

In another broad aspect the invention may be considered to be a method of treatment of Alzheimer's disease in a human subject. According to the method between about 200 mg and about 1 gram of Trans-resveratrol is administered to the subject daily. The administration is by oral ingestion and the dosage is preferably about 500 mg on a daily basis.

In still another broad aspect the invention may be considered to be a method of retarding the advance of Alzheimer's disease in a human subject. According to the invention between about 250 mg and about 1 gram of Trans-resveratrol are administered to the subject on a daily basis. The Trans-resveratrol is taken orally, preferably in dosages totaling about 500 mg daily.

The process by which the administration of Trans-resveratrol exhibits a beneficial effect on a human subject and preventing, retarding, or treating Alzheimer's disease may be described with greater clarity and particularity by reference to the accompanying drawing.

### DESCRIPTION OF THE DRAWING

The drawing FIGURE is a diagram illustrating the chemical effect in the human body of a daily administration of Trans-resveratrol in preventing, retarding or treating Alzheimer's disease in a human subject.

Resveratrol (3,4,4′-trihydroxystilbene (RV) is a constituent of grapes, mulberries and red wine with known anti-inflammatory and antioxidant activities, which are thought to be attributable to its cardio and neuro protective functions. Epidemiological studies have shown that moderate wine intake reduces the risk of Alzheimer's disease and that Trans-resveratrol is the cause of a protective effect that combats Alzheimer's disease.

The deposition of Amyloid β peptides (Aβ) in the brain is the hallmark of Alzheimer's disease and is illustrated diaphragmatically in the drawing. That is, as shown in the drawing free cholesterol (FC) in the human body reacts in a cholesterol biosynthesis process to produce cholesterol esters (CE) and at various degrees of equilibrium in different human subjects to produce Amyloid β peptides. The secretion or deposition of Amyloid β peptides in a subject is the cause, or at least a contributing factor to the development of Alzheimer's disease.

Overproduction or reduced clearance of Amyloid β peptides is closely related to the abnormal metabolism of cholesterol. High cholesterol levels in the brain increase the Amyloid β peptides deposition, which in turn increases the risk for Alzheimer's disease. On the other hand, the depletion of cellular cholesterol reduces the formation of Amyloid β peptides.

Cholesterol ester levels are directly correlated with Amyloid β peptides production. In the brain Acyl-Coenzyme A (Co-A) cholesterol acyl transferase 1 (ACAT1) exhibits a cholesterol biosynthesis. ACAT1 is a key enzyme that regulates cholesterol levels and catalyzes the formation of cholesterol esters from free cholesterol.

It is desirable for free cholesterol to efflux through the AT P binding cassette transporter (ABCA1) in a mediated process with the major apolipoprotein E as an extracellular acceptor. This process generates HDL-like apoE(E2,E3,E4)-containing particles to transport and provide membrane lipids for central nervous system cell growth, development and repair. In this process E2,E3,E4 are subsets of apolipoprotein E.

The administration to a subject of Trans-resveratrol promotes the conversion of free cholesterol to apolipoprotein E containing HDL-like particles. ACAT1, ATP binding cassette transporter and apolipoprotein E play critical roles in regulating cholesterol and Aβ peptides production and clearance in the brain. Recently ACAT1 inhibitors have been found to decrease cholesterol efflux and Aβ peptides production and dramatically reduce amyloid plaques in animals with Alzheimer's disease symptoms.

Although Trans-resveratrol has known beneficial effects in lowering plasma lipids, it was not previously known that Trans-resveratrol would have an effect on brain cellular cholesterol efflux and its association with Aβ peptides formation and clearance.

Trans-resveratrol inhibits ACAT1, increases ABCA1 mediated cholesterol efflux and reduces Aβ peptides deposition and secretion. With the administration of Trans-resveratrol some of the free cholesterol continues to be converted to 24s hydroxy cholesterol, which with a retinoid X receptor(RXR) progresses to a liver X receptor (LXRE). This in turn yields ABCA1, which is the ATP-binding cassette transporter. ABCA1 is in turn converted to apoE and Stearoyl-CoA desaturaces (SCD1). However, the administration of Trans-resveratrol changes the balance of the equilibrium cholesterol esters and free cholesterol in favor of producing and efflux of apo E which is a cholesterol efflux receptor. The apo E containing HDL-like particles transport and provide membrane lipids for growth, development and repair of brain cells.

### EXAMPLE 1

According to the present invention a human subject ingests 500 mg per day of Trans-resveratrol. The administration is by oral ingestion. The Trans-resveratrol may be taken in either a single daily dosage of 500 mg, or in multiple dosages throughout the day totaling 500 mg. The human subject in question is an individual who is elderly, that is in excess of 60 years of age, that has previously exhibited no symptoms of Alzheimer's disease.

The administration of the Trans-resveratrol in this manner results in a reduction in the APP to cholesterol-rich rafts and therefore a reduction also in Aβ peptides production. As a consequence, the individual is less prone to development of the mentally debilitating symptoms of Alzheimer's disease for a prolonged period of time while continuing to take this regimen of Trans-resveratrol.

### EXAMPLE 2

500 mg of Trans-resveratrol are administered on a daily basis to an elderly individual who has exhibited some of the preliminary indications of Alzheimer's disease, such as short-term memory lapses. With the continued administration of the 500 mg Trans-resveratrol daily dosage the advance of Alzheimer's disease is retarded so that the short-term memory lapses do not become more pronounced and so that the much more mentally debilitating effects of loss of ability to engage in meaningful conversation with others and loss of personal care abilities do not occur. The advance of Alzheimer's disease is thereby retarded.

### EXAMPLE 3

An elderly individual that exhibits significant symptoms of Alzheimer's disease, such as disorientation and loss of ability to converse in a meaningful way with others and to perform personal care activities is treated with Trans-resveratrol. Specifically, 500 mg of Trans-resveratrol are administered to the individual daily for a prolonged period of time. As a consequence, further deterioration of the mental faculties of the individual not only does not occur, but the person regains a measure of control over his or her own personal care and ability to meaningfully interact with others.

Undoubtedly, numerous variations and modifications of the invention will become readily apparent to those familiar with Alzheimer's disease. For example, the appropriate dosages of Trans-resveratrol in each individual case will vary significantly by age of the subject, degree of advancement of the symptoms of Alzheimer's disease, body weight, diet and other factors. Accordingly, the scope of the invention should not be construed as limited to the specific examples described, but rather is defined in the claims appended hereto.

1-12. (canceled)

13. A method of treating a human in need of treatment for Alzheimer's disease, comprising administering to the human Trans-resveratrol in a daily amount between about 200 mg and about 1,000 mg.

14. A method of claim 13, wherein the human has one or more symptoms of Alzheimer's disease.

15. A method of claim 13, wherein the administering occurs before the human exhibits one or more symptoms of Alzheimer's disease.

16. A method of claim 13, wherein the human is over 60 years of age.

17. A method of claim 13, wherein the administering is for a period of time sufficient to reduce the amount of amyloid plaques in the human.

18. A method of claim 13, wherein the daily amount is between 200 mg and 500 mg.

19. A method of claim 13, wherein the daily amount is between about 250 mg and about 1,000 mg.

20. A method of claim 13, wherein the daily amount is between 250 mg and 1,000 mg.

21. A method of claim 13, wherein the daily amount is between about 500 mg and about 1,000 mg.

22. A method of claim 13, wherein the daily amount is between 500 mg and 1,000 mg.

23. A method of claim 13, wherein the daily amount is between about 200 mg and about 500 mg.

24. A method of claim 13, wherein the daily amount is between 200 mg and 500 mg.

25. A method of claim 13, wherein the daily amount is about 500 mg.

26. A method of claim 13, wherein the daily amount is 500 mg.

27. A method of claim 13, wherein the administering is orally administering.

28. A method of claim 13, wherein the administering occurs once daily.

29. A method of claim 13, wherein the administering occurs multiple times daily.

30. A method of treating a human in need of treatment for Alzheimer's disease, comprising orally administering to the human Trans-resveratrol in a daily amount between 200 mg and 1,000 mg, and wherein the human has one or more symptoms of Alzheimer's disease.

31. A method of treating a human in need of treatment for Alzheimer's disease, comprising orally administering to the human Trans-resveratrol in a daily amount between 200 mg and 1,000 mg, wherein the orally administering occurs before the human exhibits one or more symptoms of Alzheimer's disease.

32. A method of claim 31, wherein the administering occurs multiple times daily.

33. A method of treating a human in need of treatment for Alzheimer's disease, comprising administering to the human a composition, in a pharmaceutically acceptable medium, consisting essentially of Trans-resveratrol in a daily amount between 200 mg and 1,000 mg.
